(19) United States
(12) Patent Application Publication
(10) Pub. No.: US 2022/0087944 A1
(43) Pub. Date: Mar. 24, 2022

(54) MULTIFUNCTIONAL (METH)ACRYLATE POLYSACCHARIDE MICROCAPSULES

(71) Applicant: Encapsys, LLC, Appleton, WI (US)

(72) Inventor: Stephen Marc Baumler, Appleton, WI (US)

(73) Assignee: Encapsys, LLC, Appleton, WI (US)

(21) Appl. No.: 17/477,644

(22) Filed: Sep. 17, 2021

Related U.S. Application Data
(60) Provisional application No. 63/080,062, filed on Sep. 18, 2020.

(51) Int. Cl. A61K 9/50

(52) U.S. Cl. CPC A61K 9/5036; A61K 9/5089; A61K 9/5026

(57) ABSTRACT

The invention describes a core shell microcapsule wherein the capsule shell is an interfacial copolymer formed of a modified polysaccharide free radical cross-linked with a multifunctional (meth)acrylate. The polysaccharide is hydrophobically modified with an adduct containing at least one unsaturated bond. The modification imparts hydrophobic character to the polysaccharide to act as an emulsifier, driving the modified polysaccharide to an oil-water interface of an encapsulation emulsion described, and creates an active bonding site for free-radical polymerization between the polysaccharide and multifunctional (meth)acrylate, thereby forming a microcapsule shell surrounding the core material.

### FIELD OF THE INVENTION

This invention relates to capsule manufacturing processes and microcapsules produced by such processes, along with improved articles of manufacture based on such microcapsules.

### DESCRIPTION OF THE RELATED ART

Various processes for microencapsulation, and exemplary methods and materials are set forth in various patents, such as Schwantes (U.S. Pat. No. 6,592,990), Nagai et al. (U.S. Pat. No. 4,708,924), Baker et al. (U.S. Pat. No. 4,166,152), Wojciak (U.S. Pat. No. 4,093,556), Matsukawa et al. (U.S. Pat. No. 3,965,033), Matsukawa (U.S. Pat. No. 3,660,304), Ozono (U.S. Pat. No. 4,588,639), Irgarashi et al. (U.S. Pat. No. 4,610,927), Brown et al. (U.S. Pat. No. 4,552,811), Scher (U.S. Pat. No. 4,285,720), Hayford (U.S. Pat. No. 4,444,699), Shioi et al. (U.S. Pat. No. 4,601,863), Kiritani et al. (U.S. Pat. No. 3,886,085), Jahns et al. (U.S. Pat. Nos. 5,596,051 and 5,292,835), Matson (U.S. Pat. No. 3,516,941), Chao (U.S. Pat. No. 6,375,872), Foris et al. (U.S. Pat. Nos. 4,001,140; 4,087,376; 4,089,802 and 4,100,103) and Greene et al. (U.S. Pat. Nos. 2,800,458; 2,800,457 and 2,730,456), among others and as taught by Herbig in the chapter entitled “Microencapsulation” in Kirk-Othmer Encyclopedia of Chemical Technology, V. 16, pages 438-463.

Other useful methods for microcapsule manufacture are: Foris et al., U.S. Pat. Nos. 4,001,140 and 4,089,802 describing a reaction between urea and formaldehyde; Foris et al., U.S. Pat. No. 4,100,103 describing reaction between melamine and formaldehyde; and British Pat. No. 2,062,570 describing a process for producing microcapsules having walls produced by polymerization of melamine and formaldehyde in the presence of a styrene sulfonic acid. Alkyl acrylate-acrylic acid copolymer capsules are taught in Brown et al., U.S. Pat. No. 4,552,811. Each patent described throughout this application is incorporated herein by reference to the extent each provides guidance regarding microencapsulation processes and materials.

Interfacial polymerization is a process wherein a microcapsule wall or polyamide, an epoxy resin, a polyurethane, a polyurea or the like is formed at an interface between two phases. Riecke, U.S. Pat. No. 4,622,267 discloses an interfacial polymerization technique for preparation of microcapsules. The core material is initially dissolved in a solvent and an aliphatic diisocyanate soluble in the solvent mixture is added. Subsequently, a nonsolvent for the aliphatic diisocyanate is added until the turbidity point is just barely reached. This organic phase is then emulsified in an aqueous solution, and a reactive amine is added to the aqueous phase. The amine diffuses to the interface, where it reacts with the diisocyanate to form polymeric polyurethane shells. A similar technique, used to encapsulate salts which are sparingly soluble in water in polyurethane shells, is disclosed in Greiner et al., U.S. Pat. No. 4,547,429. Matson, U.S. Pat. No. 3,516,941 teaches polymerization reactions in which the material to be encapsulated, or core material, is dissolved in an organic, hydrophobic oil phase which is dispersed in an aqueous phase. The aqueous phase has dissolved materials forming aminoplast (amine and aldehyde) resin which upon polymerization form the wall of the microcapsule. A dispersion of fine oil droplets is prepared using high shear agitation. Addition of an acid catalyst initiates the polycondensation forming the aminoplast resin within the aqueous phase, resulting in the formation of an aminoplast polymer which is insoluble in both phases. As the polymerization advances, the aminoplast polymer separates from the aqueous phase and deposits on the surface of the dispersed droplets of the oil phase to form a capsule wall at the interface of the two phases, thus encapsulating the core material. Urea-formaldehyde (UF), urea-resorcinol-formaldehyde (URF), urea-melamine-formaldehyde (UMF), and melamine-formaldehyde (MF), capsule formations proceed in a like manner. In interfacial polymerization, the materials to form the capsule wall are in separate phases, one in an aqueous phase and the other in an oil phase. Polymerization occurs at the phase boundary. Thus, a polymeric capsule shell wall forms at the interface of the two phases thereby encapsulating the core material. Wall formation of polyester, polyamide, and polyurea capsules also typically proceed via interfacial polymerization.

Jahns, U.S. Pat. No. 5,292,835 teaches polymerizing esters of acrylic acid or methacrylic acid with polyfunctional monomers. Specifically illustrated are reactions of polyvinylpyrrolidone with acrylates such as butanediol diacrylate or methyl methacrylate together with a free radical initiator.

Common microencapsulation processes can be viewed as a series of steps. First, the core material which is to be encapsulated is typically emulsified or dispersed in a suitable dispersion medium. This medium is typically aqueous but involves the formation of a polymer rich phase. Most frequently, this medium is a solution of the intended capsule wall material. The solvent characteristics of the medium are changed such as to cause phase separation of the wall material. The wall material is thereby contained in a liquid phase which is also dispersed in the same medium as the intended capsule core material. The liquid wall material phase deposits itself as a continuous coating about the dispersed droplets of the internal phase or capsule core material. The wall material is then solidified. This process is commonly known as coacervation.

U.S. Pat. No. 7,951,390, Jadhav et al. describes a microcapsule for agricultural applications based on various starches and starch derivatives cross-linked with vinyl monomers such as methyl methacrylate or other lower alkyl acrylates. Multifunctional methacrylates are not described, nor the robustness of multifunctional (meth)acrylate cross-linked capsules.

U.S. Pat. Pub. 20150158003 Vergallito et al. describes an acrylic polymeric shell from a multifunctional acrylic monomer and a hyperbranched polyester acrylic oligomer. Although water soluble polymers such as hydrolyzed polyvinyl alcohol, chitosan or starches are included in the polymer solution, bonding with the water-soluble polymers is not described.

In the invention, a stable capsule with tight containment can be achieved, based on cross-linking with a polysaccharide, and yet further naturally fractured and degraded after use in the intended application.

### Definitions

As used herein, reference to the term “(meth)acrylate” or “(meth)acrylic” is to be understood as referring to both the acrylate and the methacrylate versions of the specified monomer, oligomer and/or prepolymer, (for example “multifunctional (meth)acrylate” indicates that both multifunctional methacrylate and multifunctional acrylate are possible, similarly reference to alkyl esters of (meth)acrylic acid indicates that both alkyl esters of acrylic acid and alkyl esters of methacrylic acid are possible, similarly poly(meth)acrylate indicates that both polyacrylate and polymethacrylate are possible). Each alkyl moiety herein, unless otherwise indicated, can be from Ci to C8, or even from Ci to C24. Poly(meth)acrylate materials are intended to encompass a broad spectrum of polymeric materials including, for example, polyester poly(meth)acrylates, urethane and polyurethane poly(meth)acrylates (especially those prepared by the reaction of an hydroxyalkyl (meth)acrylate with a polyisocyanate or a urethane polyisocyanate), methyl cyanoacrylate, ethyl cyanoacrylate, diethylene glycol di(meth)acrylate, trimethylolpropane tri(meth)acrylate, ethylene glycol di(meth)acrylate, allyl (meth)acrylate, glycidyl (meth)acrylate, (meth)acrylate functional silicones, di-, tri- and tetra ethylene glycol di(meth)acrylate, dipropylene glycol di(meth)acrylate, polyethylene glycol di(meth)acrylate, di(penta methylene glycol) di(meth)acrylate, ethylene di(meth)acrylate, neopentyl glycol di(meth)acrylate, trimethylolpropane tri(meth)acrylate, ethoxylated bisphenol A di(meth)acrylates, bisphenol A di(meth)acrylates, diglycerol di(meth)acrylate, tetra ethylene glycol dichloroacrylate, 1,3-butanediol di(meth)acrylate, neopentyl di(meth)acrylate, trimethylolpropane tri(meth)acrylate, polyethylene glycol di(meth)acrylate and dipropylene glycol di(meth)acrylate and various multifunctional (meth)acrylates and multifunctional amine (meth)acrylates.

### SUMMARY OF THE INVENTION

The present invention teaches a microcapsule comprising a core material and a shell encapsulating the core material. The shell comprises a free radical addition product of a multifunctional (meth)acrylate cross-linked with a hydrophobically modified polysaccharide wherein the polysaccharide is characterized as having from 0.5 up to 40 mole percent of the hydroxy groups substituted with a hydrophobizing agent containing from 1 to 200 carbons. The hydrophobizing agent is selected to have at least one unsaturated bond. In embodiments, the polysaccharide is an alkenyl succinic anhydride starch selected from the group consisting of starch alkenyl succinate, starch dodecenyl succinate, starch oleic succinate, starch nonenyl succinate, or starch crotonic succinate. The alkenyl succinic anhydride modified starch has available hydroxy groups on the polysaccharide that can be further modified.

Using a suitable base, such as sodium hydroxide and a cross-linker such as epichlorhydrin, the polysaccharide can be hydrophobically modified. More particularly the hydroxy groups of the polysaccharide are modified to covalently bond to alkoxylated fatty alcohols. This increases the hydrophobicity of the polysaccharide. Useful alkoxylated fatty alcohols by way of illustration and not limitation can comprise the group selected from one or more of an ethoxylated fatty alcohol, a propoxylated fatty alcohol, an ethoxy/propoxy mixture of fatty alcohol adducts, and ethylene oxide-propylene oxide di- or tri-block copolymers. The skilled artisan will recognize that various processes for hydrophobic modification of polysaccharides can be employed such as through esterification reactions, etherification reactions or alkylation reactions.

It is desirable for the hydrophobizing agent to contain greater than one unsaturated bond or unsaturated fatty acid chain. In a further embodiment, the polysaccharide is modified with a hydrophobizing agent comprising acryloyl or methacryloyl groups, the acryloyl or methacryloyl groups undergoing an esterification reaction with the hydroxy groups. In an alternative embodiment, the polysaccharide is further hydrophobically-modified by admixture with one or more alkylene oxide.

In embodiments, alternatively, the polysaccharide can optionally be further cationically modified. The polysaccharide can be anionically-modified by admixture with chloroacetic acid or through oxidation of the polysaccharide using hypochlorite, periodate, peroxide, or the like.

The shell can comprise a free radical addition product of a multifunctional (meth)acrylate cross-linked with a polysaccharide of Formula I

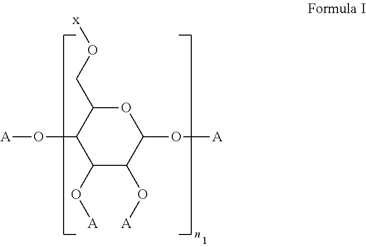

wherein n₁ is an integer from 1 to 1,000, wherein x is a terminal hydrogen or a repeating glucose unit of Formula I from 1 to 50 units, and wherein each A are independently a terminal hydrogen, or an adduct selected from the group consisting of Formula II, Formula III, Formula IV, Formula V, and Formula VI;

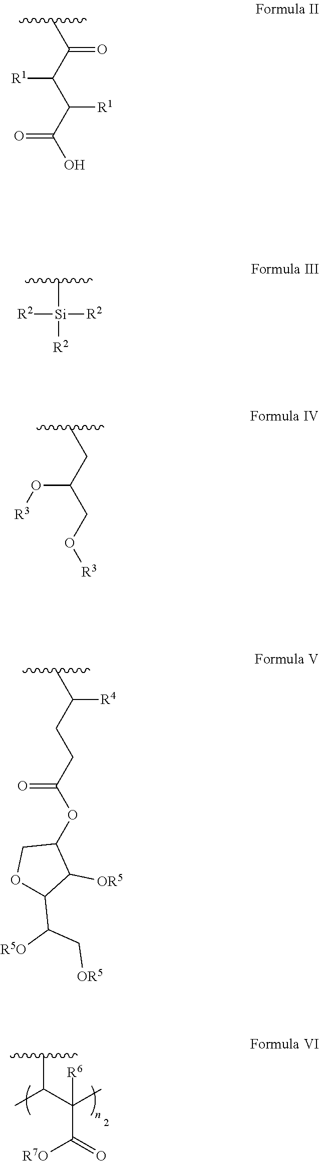

wherein each R¹ are independently a terminal hydrogen, alkyl or alkenyl, from 1 to 20 carbons, each R² are independently an alkyl, alkenyl, alkoxy, from 1 to 20 carbons or hydroxy, each R³ are independently a terminal hydrogen, alkyl or alkenyl, from 1 to 20 carbons, R⁴ is an alkyl or alkenyl, from 1 to 20 carbons, R⁵ is a terminal hydrogen, alkyl, alkenyl, from 1 to 20 carbons, or polyoxyethylene, from 1 to 40 carbons, R⁶ is a terminal hydrogen, a hydroxy, an alkoxy, alkyl, alkenyl, alkanol or alkylamine, from 1 to 20 carbons, and R⁷ is a terminal hydrogen, (meth)acrylate, alkyl, alkenyl, alkynyl, amine, amide, arenyl, carboxylate, disulfide, ester, ether, epoxide, isocyanate, or sulfide, from 1 to 20 carbons, and n₂ is an integer between 1 and 1,000.

The skillful artisan will recognize that alternative bonding sites to those shown in formulas II-VI can arise when a functional group in any of formula II-VI reacts with a nucleophilic carbon or oxygen group of the glucose moiety.

In one embodiment, the polysaccharide according to Formula 1 is octenyl succinate anhydride starch. The polysaccharide also acts as an emulsifier. Alternatively, in another embodiment, the polysaccharide according to Formula I, comprises a glucose oligomer of from 1 to 50 glucose units augmented by substitution with any of the adducts according to Formulas II, III, IV, V or VI. In the invention the shell is a copolymer of the multifunctional (meth)acrylate cross-linked with the polysaccharide which can also function as the emulsifier. The shell comprises a random copolymer of the multifunctional (meth)acrylate cross-linked with this polysaccharide emulsifier. The ratio of polysaccharide to multifunctional (meth)acrylate in the copolymer comprises a ratio of from 1:100 to 10:1 on the basis of weight.

In yet a further embodiment, the copolymer comprises a multifunctional (meth)acrylate having a vinyl group cross-linked with a hydroxyl group or carbon of the polysaccharide. In that the polysaccharides described in the invention are able to act as an emulsifier, additional emulsifier then becomes optional. The multifunctional (meth)acrylate can be selected from a monomer, oligomer or prepolymer having a greater than one ester group on average in the monomer, oligomer or prepolymer.

The core material comprises a benefit agent and, in the microcapsules, the microcapsule has a ratio of core to shell up to 99:1 or even 99.5:1, or even from 60:40 to 99:1, or even from 70:30 to 95:5, on the basis of weight. The benefit agent can be selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, antiperspirant active, emollient, humectant, exfoliant, ultraviolet absorbing agent, corrosion inhibitor, silicone oil, wax, bleach particle, fabric conditioner, malodor reducing agent, dye, optical brightener and mixtures thereof.

The invention describes a process of forming the population of microcapsules, the microcapsules comprising a core material and a shell encapsulating the core material, the shell of the microcapsules formed by an interfacial reaction between a multifunctional (meth)acrylate dispersed in an oil phase, and a polysaccharide dispersed in a water phase. Usefully, the polysaccharide can act as an emulsifier. The process comprises: a) dispersing in an oil phase a first initiator, a core material, and a multifunctional (meth)acrylate monomer, prepolymer or oligomer; b) dispersing in a water phase the polysaccharide; c) optionally adding a second initiator to the water phase; d) emulsifying the oil phase into the water phase under high shear agitation to form an oil-in-water emulsion comprising droplets of the core material and oil phase monomer dispersed in the water phase; and e) activating the initiator or initiators by heat or actinic radiation to react the multifunctional (meth)acrylate monomer, prepolymer or oligomer, and polysaccharide by free radical addition polymerization, thereby forming a polymer shell surrounding the droplets of the emulsion.

In the process of the invention, the polysaccharide comprises a moiety with an active hydroxyl or carbon and the multifunctional (meth)acrylate comprises a moiety with an unsaturated site for cross-linking to form the polymeric shell. The polysaccharide in the water phase functions as the emulsifier in the emulsifying step. Therefore, the emulsifying step is substantially free of the need for other added emulsifier.

The invention also discloses article of manufacture incorporating the microcapsules is disclosed. The article of manufacture can be selected from the group consisting of a soap, a surface cleaner, a laundry detergent, a fabric softener, a shampoo, a textile, a paper towel, an adhesive, a wipe, a diaper, a feminine hygiene product, a facial tissue, a pharmaceutical, a napkin, a deodorant, a heat sink, a foam, a pillow, a mattress, bedding, a cushion, a cosmetic, a medical device, packaging, an agricultural product, a cooling fluid, a wallboard, and an insulation.

A need exists for microcapsules having lower leakage, able to protect, retain or deliver a benefit agent to a targeted situs. Assembling robust microcapsules based at least in part on natural, renewable or sustainable components continues to be an unmet need. The present invention advances the art by teaching such microcapsules, processes of making and articles beneficially employing such microcapsules. The present invention teaches novel microcapsules based on natural components integrated into the shell by covalent bonding.

### DETAILED DESCRIPTION

The invention describes a novel core-shell microcapsule and process of preparation based on a copolymer of a modified polysaccharide cross linked with a multifunctional (meth)acrylate, along with novel articles of manufacture utilizing such microcapsule. The modified polysaccharide in the invention participates in copolymer formation and can function as an emulsifier.

The core material can be a hydrophobic/lipophilic liquid material or even a solid material. The core material serves as the encapsulated material and provides an interface for the deposition of the shell.

The shell is formed of the modified polysaccharide and multifunctional (meth)acrylate copolymer prepared in the oil phase using oil soluble radical initiators and stabilized by a polysaccharide emulsifier in the water phase, which also forms the copolymer. Formation of the shell serves to further stabilize the interface between the core material and the continuous aqueous phase by reducing the difference in surface energy.

The microcapsule is prepared by the following procedure: core material and an oil soluble acrylate are mixed (oil phase 1); core material and oil soluble radical initiators are pre-reacted (oil phase 2); water, polysaccharide emulsifier and optionally a water-soluble initiator are mixed (water phase 1); oil phase 1 and oil phase 2 are mixed after pre-reaction; water phase 1 is added to the combined oil phases and emulsified to desired size; and curing is effected, such as by heating, to form microcapsules.

Having briefly discussed the novel core-shell microcapsule and process of preparation, attention is now directed to details of the microcapsule formulation and components.

The invention is a population of microcapsules and process of forming, along with articles of manufacture comprising such microcapsules. More particularly, the invention describes a microcapsule comprising a core material and a shell encapsulating the core material, the shell comprising a free radical addition product of a multifunctional (meth)acrylate cross-linked with a polysaccharide emulsifier of Formula I,

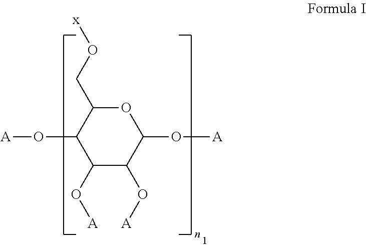

wherein n₁ is an integer from 1 to 1,000, wherein x is a terminal hydrogen or repeating glucose unit of Formula I from 1 to 50 units, and wherein each A are independently a terminal hydrogen, or an adduct selected from the group consisting of Formula II, Formula III, Formula IV, Formula V, and Formula VI;

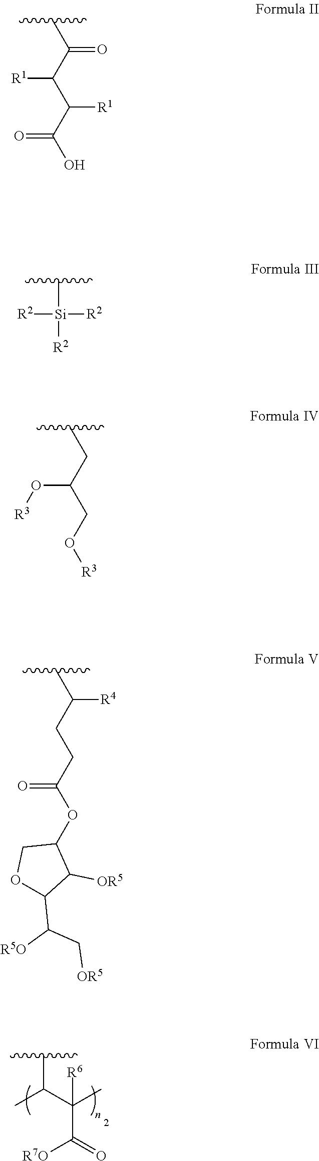

wherein each R¹ are independently a terminal hydrogen, alkyl or alkenyl, from 1 to 20 carbons, each R² are independently an alkyl, alkenyl, alkoxy, from 1 to 20 carbons or hydroxy, each R³ are independently a terminal hydrogen, alkyl or alkenyl, from 1 to 20 carbons, R⁴ is an alkyl or alkenyl, from 1 to 20 carbons, R⁵ is a terminal hydrogen, alkyl, alkenyl, from 1 to 20 carbons, or polyoxyethylene, from 1 to 40 carbons, R⁶ is a terminal hydrogen, a hydroxy, an alkoxy, alkyl, alkenyl, alkanol or alkylamine, from 1 to 20 carbons, and R⁷ is a terminal hydrogen, (meth)acrylate, alkyl, alkenyl, alkynyl, amine, amide, arenyl, carboxylate, disulfide, ester, ether, epoxide, isocyanate, or sulfide, from 1 to 20 carbons, and n₂ is an integer between 1 and 1,000.

For clarity, the wavy line in structures of Formulas II, III, IV, V and VI is intended to signify a point of attachment of the respective structure, when bonding to the structure of Formula I.

The polysaccharide is modified first or in-situ to impart changes to the hydrophilic/lipophilic properties of the native starch via a free radical addition of a multifunctional (meth)acrylate, esterified (Formula II), silylated (Formula III), or etherified with an epoxy to form the hydrophobically modified polysaccharide emulsifier.

The polysaccharide described in this invention is modified to prepare a hydrophobically modified polysaccharide useful also as the emulsifier. However, optionally, other emulsifiers can be employed. Optional emulsifiers can be anionic, cationic, non-ionic and amphoteric emulsifiers. Generally preferred emulsifiers are the cationic and non-ionic emulsifiers, particularly those having polyalkylether units, especially polyethylene oxide units, with degrees of polymerization of the alkylene ether unit of greater than about 6. Preferred emulsifiers are those which significantly reduce the interfacial tension between the aqueous phase and oil phase, and thereby reduce the tendency for droplet coalescence. In this regard, generally the emulsifiers for use in the water phase for aiding in the oil in water emulsion or dispersion will have HLB values of from 8 to 20. Emulsifiers/surfactants of lower and higher HLB values that achieve the same objective may be employed.

For many emulsifiers, hydrophobic-lipophilic balance numbers (HLB) are reported in the literature and can be a useful guide in selection of the optional additional emulsifier.

| Emulsifier | HLB value |
|---|---|
| Glycerol monostearate | 3.8 |
| Diglycerol monostearate | 5.5 |
| Tetraglycerol monostearate | 9.1 |
| Succinic acid ester of monoglycerides | 5.3 |
| Diacetyl tartaric acid ester of monoglycerides | 9.2 |
| Sodium stearoyl-2-lactylate | 21.0 |
| Sorbitan tristerate | 2.1 |
| Sorbitan monostearate | 4.7 |
| Sorbitan monooleate | 4.3 |
| Polyoxyethylene Sorbitan monostearate | 14.9 |
| Propylene glycol monostearate | 3.4 |
| Polyoxyethylene Sorbitan monooleate | 15.0 |

Typical w/o emulsifiers generally have an HLB (hydrophilic-lipophilic balance) value of 3 to 6. HLB values above about 8 generally are used to promote o/w emulsions. Optional emulsifiers of all types are suitable for use in the practice of the present invention though it is to be appreciated, and those skilled in the art will readily recognize that different systems, i.e., different oil phase compositions, will be better suited with one or more classes of emulsifiers than others.

A “glucose unit,” for purposes hereof, refers to a starting structure of a simple sugar, and is understood to refer to one or more individual units of C6H1206 monosaccharide, comprising the polysaccharide. A glucose unit is inclusive of D-glucose, L-glucose and the racemate. A glucose unit is depicted in the bracketed portion of the structure in Formula I.

The core material of the microcapsule described herein can be a lipophilic/hydrophobic liquid or even a solid material. The core material can be the intended benefit agent and the benefit agent can be the majority or minority constituent encapsulated by the microcapsules. In some cases, the benefit agent is diluted with a diluent oil from 0.01 to 99.9 weight percent of the core material. In some cases, the benefit agent can be effective even at trace quantities. A partitioning modifier, optionally, can be included to aid encapsulation and retention of the core.

In the invention, a microcapsule is formed of a modified polysaccharide and multifunctional (meth)acrylate copolymer. The term “multifunctional (meth)acrylate” is intended to encompass monomer, oligomers and prepolymers for purposes of this invention. In the process of the invention, the multifunctional (meth)acrylate copolymer is dissolved or dispersed in an oil phase containing a mixture of the core material and the selected meth(acrylate) monomer, oligomer, or prepolymer.

The multifunctional meth(acrylate) component for the cross-linking with the polysaccharide of Formula I, for purpose hereof, includes monomers, oligomers or prepolymers thereof, and can be selected from the group of multifunctional (meth)acrylate monomers consisting of ethylene glycol dimethacrylate, trimethylolpropane triacrylate, trimethylolpropane trimethacrylate, pentaerythritol tetraacrylate, tricyclodecane dimethanol dimethacrylate, 1,10 decanediol dimethacrylate, 1,6 hexanediol dimethacrylate, 1,9 nonanediol dimethacrylate, neopentyl glycol dimethacrylate, di-trimethylolpropane tetraacrylate, dipentaerythritol pentaacrylate, ethoxylated (2) bisphenol A dimethacrylate, 2,2 bis[4-(methacyloyl ethoxy) phenyl] propane, ethoxylated (3) bisphenol A diacrylate, dipropylene glycol diacrylate, ethoxylated (4) bisphenol A diacrylate, ethoxylated (4) bisphenol A dimethacrylate, 2,2 bis[4-(methacyloyl ethoxy) phenyl] propane, pentaerythritol triacrylate and mixtures thereof. The preceding monomers have greater than one ester group on average in the monomer, oligomer or prepolymer.

The multifunctional (meth)acrylate can also be selected from monomers, oligomers or prepolymers of polyethylene glycol 200 dimethacrylate, ethoxylated (9) trimethylolpropane triacrylate, 2,2 bis[4-(methacyloyl ethoxy) phenyl] propane, ethoxylated (30) BPA diacrylate, ethoxylated (15) trimethylolpropane triacrylate, ethoxylated glycerine triacrylate, ethoxylated (20) trimethylolpropane triacrylate, polyethylene glycol 400 dimethacrylate, polyethylene glycol 600 dimethacrylate, ethoxylated glycerine triacrylate, ethoxylated pentaerythritol tetraacrylate, polyethylene glycol 1000 dimethacrylate, polyethylene (200) glycol dimethacrylate, polyethylene glycol (200) diacrylate, polyethylene glycol (400) diacrylate, polyethylene glycol (600) diacrylate and tris (2-hydroxy ethyl) isocyanurate triacrylate.

Also useful multifunctional methacrylate monomer, oligomer or prepolymer can be selected from the additional group consisting of diethylene glycol dimethacrylate, ethoxylated (3) trimethylolpropane triacrylate, polypropylene glycol 400 dimethacrylate, ethoxylated (10) bisphenol A dimethacrylate, ethoxylated (10) bisphenol A diacrylate, 2,2 bis[4-(methacryloyl ethoxy) phenyl] propane, ethoxylated (4) pentaerythritol tetraacrylate, triethylene glycol dimethacrylate, 2-hydroxyl 1-3 dimethacryloxy propane, ethoxylated (6) trimethylolpropane triacrylate, ethoxylated propyleneglycol dimethacrylate, 2,2 bis[4-(methacyloyl ethoxy) phenyl] propane, and polyester (meth)acrylate.

The above listed monomers, oligomers and prepolymers are illustrative and not limiting. Multifunctional methacrylate can be selected based on having at least two functional groups reactive with the polysaccharide. The reactive functional groups can include acrylate, methacrylate, vinyl, epoxy or other unsaturated site for cross-linking with the polysaccharide of Formula I.

Optionally, the multifunctional (meth)acrylates, for example, can be blended and co-reacted with polylactic acid diacrylate or dimethacrylate, polyglycolic acid diacrylate or dimethacrylate, polycaprolactone diacrylate or dimethacrylate, or a diacrylate or a di(meth)acrylate monomer containing disulfide linkages or acetal or hemiacetal functional groups. Other useful monomers for such blending and copolymerization include polylactic acid dimethacrylate, polylactic acid diacrylate, polyglycolic acid dimethacrylate, polyglycolic acid diacrylate, polycaprolactone dimethacrylate, polycaprolactone dimethacrylate, disulfide dimethacrylate, disulfide diacrylate, or bis (2-methacryloyl) oxyethyl disulfide.

The solubility and partition coefficient of the monomers tends to control the localization of the reaction site at the interface of the oil in water droplets. In certain embodiments where the monomers are more soluble in the oil phase or phases it is possible to form a matrix capsule, though in the process of the invention, a core-shell microcapsule is preferred.

In the process of the invention a first and second oil phase is prepared and is held at a pre-reaction temperature of the selected initiator. A nitrogen blanket is preferably employed. The first oil contains the core material and oil soluble (meth)acrylate monomers, oligomers, or polymers. The second oil contains the core material and oil soluble radical initiators. Additional water phases can be employed containing a first water phase containing water- and water-soluble initiator(s), a second water phase containing polysaccharide and water, and a third water phase containing water- and water-soluble acrylates.

The two oil solutions are allowed to pre-react simultaneously or independently for a time and then combined. The mixtures are stirred and held at the pre-reaction temperature for a time sufficient to pre-react the monomers, oligomers, and polymers, to form the multifunctional (meth)acrylate copolymer. After the pre-reaction step, a water phase is added to the oil solutions.

The oil phases of the process of the invention are emulsified into the water phase, forming an oil in water emulsion. High shear agitation is used to emulsify the one or more oil phases into the water phase. The solutions are milled and heated for enough time for bonding and wall deposition to occur.

The modified polysaccharide is in the water phase and is bonded at the emulsified interface through the use of activated free radical initiators in the oil and optionally in the water phase. Bonding with the multifunctional (meth)acrylate forms a polymeric film constituting the polymer shell surrounding emulsified droplets or particles of core. This invention chemically encapsulates a hydrophobic liquid material by chemically bonding the polysaccharide emulsifier to the multifunctional (meth)acrylate through the use of free radical initiators. The initiators are energy activated, meaning free radicals are generated with heat or other energy input. Water phase initiators are available commercially, such as organic radical initiators, such as Vazo initiators, Luperox organo-peroxides, ammonium persulfate, and cerium ammonium nitrate. Oil phase initiators are available commercially, such as Vazo initiators, Luperox organo-peroxides, and the like.

The monomers of the oil phase react when in proximity in a chain growth polymerization reaction promoted by activated initiators in the immiscible water phase.

The size of the microcapsules can be controlled by adjusting the speed of agitation. Smaller size dispersions are the result of faster agitation. After a desired droplet size is attained and emulsion is stabilized with polysaccharide emulsifier, the chain growth reaction of the monomers, oligomers, or polymers with the polysaccharide emulsifier results in a membrane forming on the droplets at the interface. The resultant microcapsules are of a particle size of 0.1 to 150 microns, 0.5 to 100 microns or even from 1 to 100 microns.

The polysaccharide multifunctional (meth)acrylate membrane can be further cross-linked using a variety of chemicals (borax, ammonium persulfate, epoxy resins, phosphate salts, etc.) to improve properties of the shell component.

In the invention, the polymer shell of the capsules is modified to add hydrolysable groups to facilitate hydrolysis and biodegradation from the water side of the capsule shell. Modification of the shell with the copolymer of the invention allows the polymer shell to be more vulnerable to water-side attack. With increased hydrophilicity, the copolymers help facilitate hydrolysis and biodegradation from the water side. Surprisingly, the capsule shell is hydrolysable yet able to form a durable benefit agent delivery particle or microcapsule. In the absence of modification with polysaccharides, capsules based mostly on multifunctional (meth)acrylates generally are not hydrolysable as the shell is hydrophobic.

In microencapsulation, a core material of liquid or solid benefit agent is surrounded with a polymeric shell or alternatively embedded in a matrix of the polymer shell or a secondary polymer or gel. Release of the benefit agent is achieved through fracture, diffusion or other chemical or physical factors. In some embodiments, retention over a prolonged time period is desirable. In alternative embodiments release and further degradation through hydrolysis or biodegradation is desirable to facilitate reduction in mechanical properties and degradation with environmental aging.

Surprisingly, the invention improves the degradability of acrylate-based microcapsules, incorporating natural and bio-degradable polymer (polysaccharide) into the acrylate backbone of a multifunctional acrylate capsule. The microcapsules can be used dry or as a slurry of microcapsules, in coatings, as an additive to other materials, incorporated in or on fibers or textiles, or incorporated in or on polymeric materials, foams or other substrates. Optionally after microcapsule formation, the formed microcapsule can be isolated from the water phase or continuous phase, such as by decanting, dewatering, centrifuging, spray-drying, evaporation, freeze drying or other solvent removal or drying process.

The capsules according to the invention are useful with a wide variety of capsule contents (“core materials” or “benefit agents”) including, by way of illustration and without limitation, internal phase oils, solvent oils, phase change materials, lubricants, dyes, perfumes, fragrances, cleaning oils, polishing oils, flavorants, nutrients, sweeteners, chromogens, pharmaceuticals, fertilizers, herbicides, biological actives, scents, and the like. The microcapsule core materials can include materials which alter rheology or flow characteristics or extend shelf life or product stability. Essential oils as core materials can include, for example, by way of illustration wintergreen oil, cinnamon oil, clove oil, lemon oil, lime oil, orange oil, peppermint oil and the like. Dyes can include fluorans, lactones, indolyl red, I6B, leuco dyes, all by way of illustration and not limitation. The core material typically should be dispersible or sufficiently soluble in the capsule internal phase material namely in the internal phase oil or soluble or dispersible in the monomers or oligomers solubilized or dispersed in the internal phase oil. The core materials are preferably liquid but can be solid depending on the materials selected, and with temperatures appropriately adjusted to effect dispersion.

Useful benefit agents or core materials include perfume raw materials, such as alcohols, ketones, aldehydes, esters, ethers, nitriles, alkenes, fragrances, fragrance solubilizers, essential oils, phase change materials, lubricants, colorants, cooling agents, preservatives, antimicrobial or antifungal actives, herbicides, antiviral actives, antiseptic actives, biological actives, deodorants, emollients, humectants, exfoliants, ultraviolet absorbing agents, self-healing compositions, corrosion inhibitors, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, dyes, brighteners, antibacterial actives, antiperspirant actives, cationic polymers and mixtures thereof. Phase change materials useful as core materials can include, by way of illustration and not limitation, paraffinic hydrocarbons having 13 to 28 carbon atoms, various hydrocarbons such n-octacosane, n-heptacosane, n-hexacosane, n-pentacosane, n-tetracosane, n-tricosane, n-docosane, n-heneicosane, n-eicosane, n-nonadecane, octadecane, n-heptadecane, n-hexadecane, n-pentadecane, n-tetradecane, n-tridecane. Phase change materials can alternatively, optionally in addition include crystalline materials such as 2,2-dimethyl-1,3-propanediol, 2-hydroxymethyl-2-methyl-1, 3-propanediol, acids of straight or branched chain hydrocarbons such as eicosanoic acid and esters such as methyl palmitate, fatty alcohols and mixtures thereof. Blends of capsule populations can be useful, such as with differing sets of benefit agent, or even different wall formulations.

A partitioning modifier can optionally also be included as a constituent of the microcapsule core. The partitioning modifier can be the same material as the oil phase or diluent or can be different. The partitioning modifier can be selected from a larger group and can be further selected from the group consisting of oil soluble materials that have a C log P greater than from about 4, or from about 5, or from about 7, or even from about 11 and/or materials that also have a density higher than 1 gram per cubic centimeter.

Deposition aids can include poly (acrylamide-co-diallyldimethylammonium chloride, poly (diallyldimethylammonium chloride, polyethylenimine, cationic polyamine, poly [(3-methyl-1-vinylimidazolium chloride)-co-(1-vinylpyrrolidone)], copolymer of acrylic acid and diallyldimethylammonium chloride, cationic guar, guar gum, an organopolysiloxane such as described in US Publication 20150030557, incorporated herein by reference. In a further embodiment, the above-described microcapsules can comprise a deposition aid, and in a further aspect the deposition aid coats the outer surface of the shell of the microcapsule. Deposition aids can be coated onto capsules or covalently bonded, employing functional groups to effect linkage as generally described in Universidade do Minho, WO 2006117702; Gross et al., US 20170296440; and Devan Micropolis, US 20080193761

In a further aspect the deposition aid can comprise a material selected from the group consisting of poly(meth)acrylate, poly(ethylene-maleic anhydride), polyamine, wax, polyvinylpyrrolidone, polyvinylpyrrolidone co-polymers, polyvinylpyrrolidone-ethyl acrylate, polyvinylpyrrolidone-vinyl acrylate, polyvinylpyrrolidone methacrylate, polyvinylpyrrolidone-vinyl acetate, polyvinyl acetal, polyvinyl butyral, polysiloxane, poly(propylene maleic anhydride), maleic anhydride derivatives, co-polymers of maleic anhydride derivatives, polyvinyl alcohol, styrene-butadiene latex, gelatin, gum Arabic, carboxymethyl cellulose, carboxymethyl hydroxyethyl cellulose, hydroxyethyl cellulose, other modified celluloses, sodium alginate, chitosan, casein, pectin, modified starch, polyvinyl acetal, polyvinyl butyral, polyvinyl methyl ether/maleic anhydride, polyvinyl pyrrolidone and its co polymers, poly(vinyl pyrrolidone/methacrylamidopropyl trimethyl ammonium chloride), polyvinylpyrrolidone/vinyl acetate, polyvinyl pyrrolidone/dimethylaminoethyl methacrylate, polyvinyl amines, polyvinyl formamides, polyallyl amines and copolymers of polyvinyl amines, polyvinyl formamides, and polyallyl amines and mixtures thereof.

In a yet further aspect, the deposition aid comprises a material selected from the group consisting of poly(meth)acrylates, poly(ethylene-maleic anhydride), polyamine, polyvinylpyrrolidone, polyvinylpyrrolidone-ethyl acrylate, polyvinylpyrrolidone-vinyl acrylate, polyvinylpyrrolidone methacrylate, polyvinylpyrrolidone-vinyl acetate, polyvinyl acetal, polysiloxane, poly(propylene maleic anhydride), maleic anhydride derivatives, co-polymers of maleic anhydride derivatives, polyvinyl alcohol, carboxymethyl cellulose, carboxymethyl hydroxyethyl cellulose, hydroxyethyl cellulose, polyvinyl methyl ether/maleic anhydride, polyvinylpyrrolidone/vinyl acetate, polyvinyl pyrrolidone/dimethylaminoethyl methacrylate, polyvinyl amines, polyvinyl formamides, polyallyl amines and copolymers of polyvinyl amines, polyvinyl formamides, and polyallyl amines and mixtures thereof.

The microcapsules of the invention can be incorporated dry, as an aqueous slurry, as a coating or as a gel into a variety of commercial products to yield novel and improved articles of manufacture, including incorporation into or onto foams, mattresses, bedding, cushions, added to cosmetics or to medical devices, incorporated into or onto packaging, dry wall, construction materials, heat sinks for electronics, cooling fluids, incorporated into insulation, used with lotions, incorporated into gels including gels for coating fabrics, automotive interiors, and other structures or articles, including clothing, footwear, personal protective equipment and any other article where use of the improved capsules of the invention is deemed desirable. The articles of manufacture can be selected from the group consisting of a soap, a surface cleaner, a laundry detergent, a fabric softener, a shampoo, a textile, a paper towel, an adhesive, a wipe, a diaper, a feminine hygiene product, a facial tissue, a pharmaceutical, a napkin, a deodorant, a foam, a pillow, a mattress, bedding, a cushion, a cosmetic, a medical device, an agricultural product, packaging, a cooling fluid, a wallboard, and insulation.

The microcapsules protect and separate the core material such as phase change material or fragrance or other core material or benefit agent, keeping it separated from the external environment. This facilitates design of distinct and improved articles of manufacture. The microcapsules facilitate improving flowability of encapsulated materials enhancing ease of incorporation into or onto articles such as foams, gels, textiles, various cleaners, detergents or fabric softeners. The microcapsules can be used neat, or more often blended into coatings, gels or used as an aqueous slurry or blended into other articles to form new and improved articles of manufacture. For example, with phase change benefit agents, the microcapsules help preserve the repeated activity of the phase change material and retain the phase change material to prevent leakage or infusion into nearby components when isolation of the microcapsules is desired, yet promote eventual degradation of such encapsulates or portions of the articles of manufacture.

### EXAMPLES

In the following examples, the abbreviations correspond to the following materials:

TABLE 1

| Name | Company/City | Chemical Description |
|---|---|---|
| CD9055 | Sartomer Company, | Acidic Acrylate Adhesion Promoter |
| | Exton, PA | |
| CN2262 | Sartomer Company, | Polyester acrylate oligomer |
| | Exton, PA | |
| CN975 | Sartomer Company, | Hexafunctional urethane acrylate ester |
| | Exton, PA | |
| SELVOL | Sekisui Specialty Chemicals, | Polyvinyl alcohol |
| 540 | Dallas, TX | |
| SR206 | Sartomer Company, | Ethylene glycol dimethacrylate |
| | Exton, PA | |
| SR252 | Sartomer Company, | Polyethylene glycol dimethacrylate |
| | Exton, PA | |
| SR350 | Sartomer Company, | Trimethylolpropane trimethacrylate |
| | Exton, PA | |
| SR415 | Sartomer Company, | Ethoxylated (20) Trimethylolpropane |
| | Exton, PA | triacrylate |
| SR454 | Sartomer Company, | Ethoxylated (3) Trimethylolpropane |
| | Exton, PA | triacrylate |
| TBAEMA | NovaSol North America Inc., | 2-(tert-butylamino) ethyl methacrylate |
| | Stoney Creek, ON, Canada | |
| V50 | Wako Specialty Chemicals, | 2,2′-azobis (2-methylpropionamidine) |
| | Richmond, VA | dihydrochloride |
| Vazo 67 | Chemours Company, | 2,2′-azobis (2-methylbutyronitrile) |
| | Wilmington, DE | |
| Vazo 88 | Chemours Company, | 1,1′-azobis cyclohexanecarbonitrile |
| | Wilmington, DE | |
| Luperox | Arkema | Benzoyl peroxide |
| A98 | King of Prussia, PA | |
| Captex | Abitec, | Caprylic/capric triglyceride |
| 355 | Columbus, OH | |
| PLA-DM | | Polylactic acid dimethacrylate |
| HI-CAP | Ingredion, | Starch sodium octenyl succinate |
| 100 | Westchester, IL | |
| PURITY | Ingredion, | Starch sodium octenyl succinate |
| GUM 2000 | Westchester, IL | |
| IM- | Ingredion, | Starch sodium octenyl succinate |
| CREAMER | Westchester, IL | |
| CAPSUL | Ingredion, | Starch sodium octenyl succinate |
| | Westchester, IL | |
| GLOBE 10 | Ingredion, | Maltodextrin (DE 10) |
| | Westchester, IL | |

The core oil used in the examples is an equal part blend of Captex 355 (triglycerides of caprylic/capric acid) and fragrance blend. The fragrance blend employed in the examples is an equal part blend of benzyl acetate, octanal, linalool, 2,6-dimethyl7-octen-2-ol, isobornyl acetate, linalyl acetate, butylphenyl methylpropional, isoamyl salicylate, and hexyl salicylate.

### Example 1—Comparative Microencapsulation Process Illustrating Use of Polyvinyl Alcohol

Oil phase solution I was prepared containing 15 g of the core oil and 10.0 g of SR206. Oil phase solution II, containing 100 g of the core oil and 0.3 g of each oil phase initiator (Vazo-67 and Vazo-88), was well mixed in a jacketed steel reactor stirring at 390 rpm. Under a nitrogen blanket of 250 cc/min, oil phase solution II was pre-heated from 35° C. to 70° C. and held at 70° C. for 45 minutes and cooled from 70° C. to 50° C. over 45 minutes. Oil phase solution I was well mixed and added to oil phase solution II after returning to 50° C. Oil phase solution I and oil phase solution II are well mixed and allowed to react for 10 minutes prior to the addition of the water phase solution. The water phase solution containing 275 g of water, 0.3 g of V-50, and 45 g of a 5 wt % by solids solution of SELVOL 540 was mixed at 500 rpm. The water phase was pre-heated to 50° C. prior to addition to the oil phase. The water phase was added to the oil phase and mixing speed was increased to approximately 1700 rpm. After one hour, the batch temperature was increased from 50° C. to 70° C. and held at 70° C. for 4 hours, then increased from 70° C. to 95° C. and held at 95° C. to complete reaction before cooling to room temperature naturally.

### Examples 2-5—Microencapsulation Process Illustrating Use of Different OSA-Modified Starches of Formula II

Examples 2-5 were prepared as according to example 1, replacing the SELVOL 540 with 35 g of the starch indicated in table 2.

### Examples 6-11—Microencapsulation Process Illustrating Use of Single Component Multifunctional (Meth)Acrylates

Examples 6-11 were prepared according to example 1, replacing SELVOL 540 with 35 g of HI-CAP 100 and SR206 with the Acrylate according to table 2.

### Examples 12-15—Microencapsulation Process Illustrating Use of Varied Amounts of Multifunctional (Meth)Acrylates

Examples 12-15 were prepared according to example 1, replacing SELVOL 540 with 35 g of HI-CAP 100 and SR206 with the Acrylate in the quantities shown in table 2.

### Examples 16-18—Microencapsulation Process Illustrating Use of Multi-Component Multifunctional (Meth)Acrylates

Examples 16-18 were prepared according to example 1, replacing SELVOL 540 with 35 g of HI-CAP 100 and SR206 with the Acrylate in the quantities shown in table 2. An additional multifunctional acrylate (CD9055) was added at the time of Acrylate I addition in the quantities specified in table 2.

### Example 19—Microencapsulation Process Illustrating Use of a Peroxide Initiator

Example 19 was prepared according to example 18, replacing the Oil phase solution II initiators with 0.7 g of Luperox A98.

### Example 20—Hydrophobic Modification of Polysaccharide Illustrating Formula III

The polysaccharide emulsifier is prepared by mixing 45 g of Globe 10 from corn with 400 g water. After maltodextrin is dissolved, 4 g of caustic soda solution (21.5%) is added to the maltodextrin solution. An equal parts silane (7-octyltrimethoxysilane) and methanol solution is prepared and added dropwise to the stirring maltodextrin caustic solution to a total of 1 percent silane by weight. After the silane alcohol solution is added, the solution is heated at 40° C. for two hours. The polysaccharide emulsifier is freeze dried and Soxhlet extracted using 2-propanol for 24 hours. After extraction, the polysaccharide emulsifier is dried under vacuum prior to use.

### Example 21—Microencapsulation Process Illustrating Use of a Hydrophobically Modified Polysaccharide According to Formula III

Example 21 is prepared according to example 1, replacing the SELVOL 540 with 35 g of the hydrophobically modified starch described in example 20.

### Example 22—Hydrophobic Modification of Polysaccharide Illustrating Formula IV

The hydrophobically modified polysaccharide is prepared by mixing 25 g of Globe 10 from corn, 36.5 g of octyl/decyl glycidyl ether, 141 g water, and 50 g of 21.5% caustic soda. After solution is well mixed, the solution is then heated at 40° C. for 18 hours, cooled to room temperature and neutralized to pH 7 using a 2.5M hydrochloric acid solution. The hydrophobically modified polysaccharide is freeze dried and Soxhlet extracted using 2-propanol for 24 hours. After extraction, the modified polysaccharide is dried under vacuum prior to use.

### Example 23—Microencapsulation Process Illustrating Use of a Hydrophobically Modified Polysaccharide According to Formula IV

Example 23 is prepared according to example 1, replacing the SELVOL 540 with 35 g of the hydrophobically modified starch described in example 22.

### Example 24—Hydrophobic Modification of Polysaccharide Illustrating Formula V

The hydrophobically modified polysaccharide was prepared by mixing 20 g of Globe 10 from corn, and 400 g water. A coarse emulsion was prepared by mixing 15 g of the maltodextrin solution with 10 g of Tween 80 under high shear mixing at 10,000 rpm using an IKA Ultraturrax T25 with 18 G head for 3 minutes. The maltodextrin solution was heated to 60° C. and 1.0 g of potassium persulfate was added. 30 minutes after the addition of potassium persulfate, the Tween 80 and maltodextrin emulsion was added dropwise to the maltodextrin solution over a total of five minutes. The maltodextrin solution was reacted for a total of four hours at 60° C., then cooled to room temperature naturally. The hydrophobically modified polysaccharide was freeze dried and Soxhlet extracted using 2-propanol for 24 hours. After extraction, the modified polysaccharide was dried under vacuum prior to use.

### Example 25—Microencapsulation Process Illustrating Use of a Hydrophobically Modified Polysaccharide According to Formula V

Example 25 is prepared according to example 18, adding 0.1 g of CD9055, replacing the HI-CAP 100 with 21 g of the hydrophobically modified starch described in example 24, and less 121 g water.

### Example 26—Microencapsulation Process Illustrating Use of a Hydrophobically Modified Polysaccharide According to Formula V

Example 26 is prepared according to example 25, replacing 50% of the Tween 80 with Tween 85 in example 24.

### Example 27—Hydrophobic Modification of Polysaccharide Illustrating Formula VI

The hydrophobically modified polysaccharide was prepared by mixing a first water phase solution, WPI, containing 250 g of water with 1 g of water phase initiator (V50). WPI is added to a jacketed beaker reactor at 40° C. and mixed under a nitrogen blanket at 100 cc/min. WPI was then heated from 40° C. to 75° C. over 45 minutes and held for an additional 45 minutes. A second water phase solution, WPII, was prepared by mixing 20 g of maltodextrin (DE 4-7, Sigma Aldrich) and 100 g water. WPII is preheated to 60° C., 30 minutes prior to the addition to WPI. WPII was added to WPI after WPI completed the above heating steps and allowed to react for 30 minutes prior to the addition of a third water phase, WPIII. WPIII was prepared by mixing 75 g of water, 10 g of SR415, and 3 g of TBAEMA. WPIII was added to the reactor dropwise over 60 minutes. After addition of WPIII, the solution was heated from 75° C. to 95° C. over four hours and held at 95° C. for an additional six hours before cooling to room temperature naturally. The solution is then sieved through a No. 120 sieve prior to use for microencapsulation.

### Example 28—Microencapsulation Process Illustrating Use of a Hydrophobically Modified Polysaccharide According to Formula V

Example 28 is prepared according to example 18, replacing the HI-CAP 100 and water with a solution containing 165 g of the hydrophobically modified polysaccharide according to example 27 (11.9% solids by weight).

TABLE 2

| | | | Starch | Acrylate | Acrylate | Acrylate | Acrylate |
|---|---|---|---|---|---|---|---|
| Example | Formula | Starch | (g) | I | I (g) | II | II (g) |
| 1 | | PVOH 540 | 0 | SR206 | 10 | — | — |
| 2 | II | HI-CAP 100 | 35 | SR206 | 10 | — | — |
| 3 | II | Purity Gum | 35 | SR206 | 10 | — | — |
| 4 | II | N-Creamer | 35 | SR206 | 10 | — | — |
| 5 | II | Capsul | 20 | SR206 | 10 | — | — |
| 6 | II | HI-CAP 100 | 35 | SR252 | 10 | — | — |
| 7 | II | HI-CAP 100 | 35 | SR350 | 10 | — | — |
| 8 | II | HI-CAP 100 | 35 | SR454 | 10 | — | — |
| 9 | II | HI-CAP 100 | 35 | CD9055 | 10 | — | — |
| 10 | II | HI-CAP 100 | 35 | CN2262 | 10 | — | — |
| 11 | II | HI-CAP 100 | 35 | CN975 | 10 | — | — |
| 12 | II | HI-CAP 100 | 35 | CN975 | 5 | — | — |
| 13 | II | HI-CAP 100 | 35 | CN975 | 1 | — | — |
| 14 | II | HI-CAP 100 | 35 | CN2203 | 5 | — | — |
| 15 | II | HI-CAP 100 | 35 | CN2203 | 1 | — | — |
| 16 | II | HI-CAP 100 | 35 | SR415 | 6 | CD9055 | 0.4 |
| 17 | II | HI-CAP 100 | 35 | SR454 | 7 | CD9055 | 0.4 |
| 18 | II | HI-CAP 100 | 35 | SR206 | 5 | CD9055 | 0.4 |
| 19 | II | HI-CAP 100 | 35 | SR206 | 5 | CD9055 | 0.4 |
| 21 | III | Example 20 | 35 | SR206 | 5 | CD9055 | 0.5 |
| 23 | IV | Example 22 | 35 | SR206 | 5 | CD9055 | 0.5 |
| 25 | V | Example 24 | 21 | SR206 | 5 | CD9055 | 0.5 |
| 26 | V | Example 24 | 21 | SR206 | 5 | CD9055 | 0.5 |
| 28 | VI | Example 27 | 20 | SR206 | 5 | CD9055 | 0.5 |

### Test Methods

Several test methodologies were performed on the microcapsules within the present invention. These test methods were for determining the particle size, free benefit agent, and leakage in a hexane and ethanol solution. Test results are shown in table 3.

### Batch Solids

The percent solids of the microcapsule batch were measured using a microwave and infrared moisture and solids analyzer (CEM Smart 6).

### Median Volume Weighted Particle Size

The volume-weighted median particle size of the microcapsules is measured using an Accusizer 780A, made by Particle Sizing Systems, Santa Barbara Calif., or equivalent. The instrument is calibrated from 0 to 300 μm (micrometer or micron) using particle size standards (as available from Duke/Thermo-Fisher-Scientific Inc., Waltham, Mass., USA). Samples for particle size evaluation are prepared by diluting about 0.5 g of microcapsule slurry in about 10 g of de-ionized water. This dilution is further diluted using about 1 g of the initially diluted solution in about 20 g of water. Approximately 1 g of the most dilute sample is injected into the Accusizer, and the testing initiated using the autodilution feature. The Accusizer should read more than 8,500 counts/second. If the counts are below 8,500 additional sample is added. The sample is autodiluted until below 9,200 counts/second was measured, then particle counting, and size analysis is initiated. After 2 minutes of testing, the Accusizer displays the median volume-weighted particle size. Particle sizes stated herein are on a volume weighted basis and are to be understood as median volume weighted particle size, ascertainable by the above procedure.

Percent Free Oil after Microencapsulation

Characterization of free oil in microcapsule suspension: 0.4-0.5 g of the microcapsule suspension is massed and mixed with 10 ml of hexane. The sample is mixed by vortexing at 3000 rpm for 10 seconds to leach the free oil from the microcapsule suspension and set aside for no more than one minute. An aliquot is removed from the hexane layer and filtered through a 0.45 μm syringe filter. The concentration of oil in the hexane is measured using an Agilent 7800 Gas Chromatograph (GC), Column: ZB-1 HT (10 meter×0.32 mm×0.25 μm), Temp: 50° C. for 1 minute then heat to 270° C. @ 10° C./min, Injector: 275° C., Detector: 325° C., 2 μl injection.

Percent Leakage of Oil after 1 Week in Liquid Fabric Enhancer (LFE)

Characterization of the percent free oil after 1 week in liquid fabric enhancer: The percent activity of the microcapsule slurry is calculated as the grams of benefit agent divided by grams of microcapsule slurry. The mass of the slurry needed for testing is then calculated as 1.5 divided by the percent activity. 50 g of Downy Fabric Softener is added to a glass jar. The appropriate mass of slurry is massed and placed in the jar containing liquid fabric enhancer under stirring until homogenized. The jar is capped and placed in an oven at 35° C. for one week. After one week the amount of free oil is measured. 0.4-0.5 g of the microcapsule suspension is massed, mixed with 2 mL RO water, and vortexed at 1,000 rpm for 60 second. 10 ml of hexane is added and vortexed at 1,000 rpm for 60 seconds. The sample is allowed to rest 30 minutes. An aliquot is removed from the hexane layer and filtered through a 0.45 μm syringe filter. The concentration of oil in the hexane is measured using an Agilent 7800 Gas Chromatograph (GC), Column: ZB-1 HT (10 meter×0.32 mm×0.25 μm), Temp: 50° C. for 1 minute then heat to 270° C. @ 10° C./min, Injector: 275° C., Detector: 325° C., 2 μl injection.

### Determination of Release Properties of Oil (MT190)

Characterization of the release properties of the core oil were measured using the CIPAC MT190 test method. The percent free CAPTEX 355 after a one-hour extraction was normalized to the total concentration of CAPTEX 355 contained in the microcapsule slurry and reported in table 3.

TABLE 3

| | % | Size | % Benefit | Free Oil | LFE | MT190(60) |
|---|---|---|---|---|---|---|
| Example | Solids | (μm) | Agent | (RA) | (RA) | % Actives |
| 1 | 30.3 | 20.8 | 13.6 | 0.1 | 18.9 | 52.8 |
| 2 | 46.4 | 18.1 | 16.6 | 0.2 | 37.9 | 24.2 |
| 3 | 40.1 | 18.5 | 13.3 | 0.1 | 36.0 | 10.6 |
| 4 | 41.9 | 16.4 | 15.0 | 0.1 | 38.8 | 6.9 |
| 5 | 42.4 | 127.5 | 16.7 | 4.5 | 46.5 | 37.6 |
| 6 | 42.7 | 26.6 | 15.3 | 2.6 | 37.8 | 54.1 |
| 7 | 39.8 | 20.3 | 14.2 | 0.5 | 22.8 | 28.4 |
| 8 | 45.2 | 20.3 | 16.1 | 0.4 | 19.6 | 22.0 |
| 9 | 38.3 | 43.4 | 13.7 | 2.1 | 42.3 | 19.2 |
| 10 | 79.3 | 19.8 | 28.4 | 0.2 | 33.3 | 49.9 |
| 11 | 37.4 | 23.9 | 13.4 | 0.5 | 51.7 | 28.1 |
| 12 | 44.1 | 24.7 | 16.3 | 0.2 | 25.0 | 24.8 |
| 13 | 41.1 | 26.3 | 15.6 | 1.3 | 34.9 | 34.2 |
| 14 | 41.2 | 21.8 | 15.2 | 1.8 | 32.8 | — |
| 15 | 49.8 | 25.1 | 18.8 | 1.5 | 34.5 | — |
| 16 | 38.5 | 25.1 | 14.0 | 8.8 | 25.8 | — |
| 17 | 39.4 | 15.4 | 14.3 | 9.5 | 16.6 | — |
| 18 | 38.4 | 14.3 | 15.4 | 45.7 | 29.4 | — |
| 19 | 38.6 | 16.2 | 14.2 | 6.9 | 27.1 | — |
| 25 | 49.3 | 21.6 | 19.9 | 15.4 | 63.6 | — |
| 26 | 46.4 | 19.6 | 18.7 | 12.9 | 53.2 | — |
| 28 | 52.8 | 9.5 | 24.8 | 0.5 | 27.1 | — |

All documents cited in the specification herein are, in relevant part, incorporated herein by reference for all jurisdictions in which such incorporation is permitted. The citation of any publication is for its disclosure prior to the filing date and should not be construed as an admission that such publication is prior art or that the present invention is not entitled to antedate such publication by virtue of prior invention. To the extent that any meaning or definition of a term in this document conflicts with any meaning or definition of the same term in a document incorporated by reference, the meaning or definition assigned to that term in this document shall govern. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. No unclaimed language should be deemed to limit the invention in scope. Any statements or suggestions herein that certain features constitute a component of the claimed invention are not intended to be limiting unless reflected in the appended claims.

The principles, preferred embodiments, and modes of operation of the present invention have been described in the foregoing specification. The invention, which is intended to be protected herein, however, is not to be construed as limited to the particular forms disclosed, since these are to be regarded as illustrative rather than restrictive variations and charges can be made by those skilled in the art without departing from the spirit and scope of the invention.

What we claim is:

1. A microcapsule comprising a core material and a shell encapsulating the core material, wherein the shell comprises a free radical addition product of a multifunctional (meth)acrylate cross-linked with a hydrophobically modified polysaccharide wherein the polysaccharide is characterized as having hydroxy groups of which from 0.5 up to 40 mole percent of the hydroxy groups of the polysaccharide are substituted with a hydrophobizing agent containing from 1 to 200 carbons.

2. The microcapsule according to claim 1 wherein the hydrophobizing agent has at least one unsaturated bond.

3. The microcapsule according to claim 2 wherein the polysaccharide is an alkenyl succinic anhydride starch selected from the group consisting of starch alkenyl succinate, starch dodecenyl succinate, starch oleic succinate, starch nonenyl succinate, or starch crotonic succinate.

4. The microcapsule according to claim 1 wherein the polysaccharide hydroxy groups are modified with an alkoxylated fatty alcohol comprising the group selected from one or more of an ethoxylated fatty alcohol, a propoxylated fatty alcohol, an ethoxy/propoxy mixture of fatty alcohol adducts, and ethylene oxide-propylene oxide di- or tri-block copolymer.

5. The microcapsule according to claim 1 wherein the hydrophobizing agent contains greater than one unsaturated bond or unsaturated fatty acid chains.

6. The microcapsule according to claim 1 wherein the polysaccharide is modified with a hydrophobizing agent comprising acryloyl or methacryloyl groups, the acryloyl or methacryloyl groups undergoing an esterification reaction with the hydroxy groups.

7. The microcapsule according to claim 1 wherein the polysaccharide is further hydrophobically-modified by admixture with one or more alkylene oxide.

8. The microcapsule according to claim 1 wherein the polysaccharide is further cationically-modified.

9. The microcapsule according to claim 1 wherein the polysaccharide is further anionically-modified by admixture with chloroacetic acid or through oxidation of the polysaccharide.

10. A microcapsule according to claim 1 wherein the shell comprises a free radical addition product of a multifunctional (meth)acrylate cross-linked with a hydrophobically modified polysaccharide according to Formula I 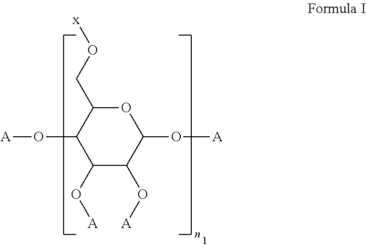
wherein n₁ is an integer from 1 to 1,000, wherein x is a terminal hydrogen or repeating glucose unit of Formula I from 1 to 50 units, and wherein each A are independently a terminal hydrogen, or an adduct selected from the group consisting of Formula II, Formula III, Formula IV, Formula V, and Formula VI, 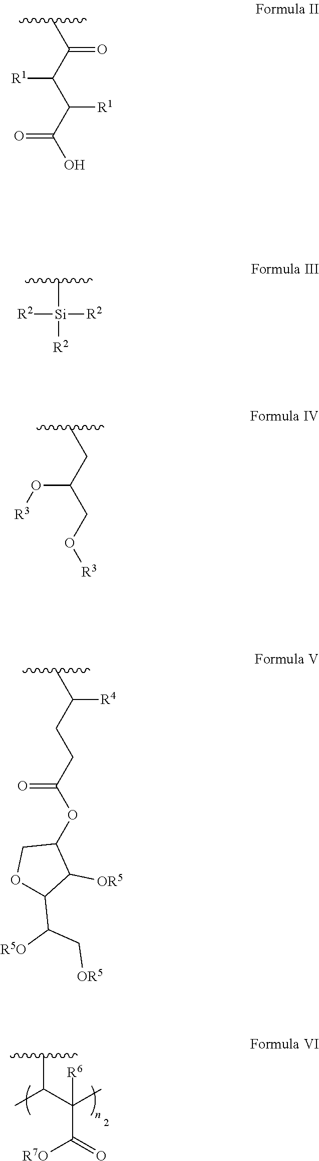
wherein each R¹ are independently a terminal hydrogen, alkyl or alkenyl, from 1 to 20 carbons, each R² are independently an alkyl, alkenyl, alkoxy, from 1 to 20 carbons or hydroxy, each R³ are independently a terminal hydrogen, alkyl or alkenyl, from 1 to 20 carbons, R⁴ is an alkyl or alkenyl, from 1 to 20 carbons, R⁵ is a terminal hydrogen, alkyl, alkenyl, from 1 to 20 carbons, or polyoxyethylene, from 1 to 40 carbons, R⁶ is a terminal hydrogen, a hydroxy, an alkoxy, alkyl, alkenyl, alkanol or alkylamine, from 1 to 20 carbons, and R⁷ is a terminal hydrogen, (meth)acrylate, alkyl, alkenyl, alkynyl, amine, amide, arenyl, carboxylate, disulfide, ester, ether, epoxide, isocyanate, or sulfide, from 1 to 20 carbons, and n₂ is an integer from 1 to 1,000.

11. The microcapsule according to claim 1 wherein the shell is a copolymer of the multifunctional (meth)acrylate cross-linked with the polysaccharide.

12. The microcapsule according to claim 1 wherein the shell comprises a random copolymer of the multifunctional (meth)acrylate cross-linked with the polysaccharide, the ratio of polysaccharide to multifunctional (meth)acrylate in the copolymer comprising a ratio of from 1:100 to 1:1 on the basis of weight.

13. The microcapsule according to claim 10 wherein the shell comprises a random copolymer of the multifunctional (meth)acrylate cross-linked with the polysaccharide, and on an AGU basis the ratio of moles of acrylate per AGU of starch is at most 1500 moles acrylate per AGU of starch.

14. The microcapsule according to claim 1 wherein the copolymer comprises a multifunctional (meth)acrylate having a vinyl group cross-linked with a hydroxyl group, carbon, or (meth)acryloyl of the polysaccharide.

15. The microcapsule according to claim 1 wherein the multifunctional (meth)acrylate comprises a monomer, oligomer or prepolymer having a greater than one ester group on average in the monomer, oligomer or prepolymer, and can be selected from ethylene glycol dimethacrylate, polyethylene glycol dimethacrylate, ethylidene dimethacrylate, trimethylolpropane triacrylate, trimethylolpropane trimethacrylate, pentaerythritol tetraacrylate, tricyclodecane dimethanol dimethacrylate, 1,10 decanediol dimethacrylate, 1,6 hexanediol dimethacrylate, 1,9 nonanediol dimethacrylate, neopentyl glycol dimethacrylate, di-trimethylolpropane tetraacrylate, dipentaerythritol pentaacrylate, ethoxylated (2) bisphenol A dimethacrylate, 2,2 bis[4(methacyloyl ethoxy) phenyl] propane, ethoxylated (3) bisphenol A diacrylate, dipropylene glycol diacrylate, ethoxylated (4) bisphenol A diacrylate, ethoxylated (4) bisphenol A dimethacrylate, 2,2 bis[4-(methacyloyl ethoxy) phenyl] propane, polyethylene glycol trimethylolpropane tri(meth)acrylate, polyester acrylate urethane acrylate and pentaerythritol triacrylate.

16. The microcapsule according to claim 1 wherein the microcapsule has a ratio of core to shell up to 99:1 or even 99.5:1, on the basis of weight.

17. The microcapsule according to claim 1 wherein the core material comprises a benefit agent.

18. The microcapsule according to claim 17 wherein the benefit agent can be selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, antiperspirant active, emollient, humectant, exfoliant, ultraviolet absorbing agent, corrosion inhibitor, silicone oil, wax, bleach particle, fabric conditioner, malodor reducing agent, dye, optical brightener and mixtures thereof.

19. A process of forming a population of microcapsules, the microcapsules comprising a core material and a shell encapsulating the core material, the shell of the microcapsules formed by an interfacial reaction between a multifunctional (meth)acrylate dispersed in an oil phase, and a polysaccharide dispersed in a water phase, the process comprising:
a. dispersing one or more in oil phases a first initiator, a core material, and a multifunctional (meth)acrylate monomer, prepolymer or oligomer;
b. dispersing in a water phase the polysaccharide, and an optional second initiator;
c. emulsifying the one or more oil phases into the water phase under high shear agitation to form an oil-in-water emulsion comprising droplets of the core material and oil phase monomer, prepolymer or oligomer dispersed in the water phase;
d. and activating the initiator or initiators by heat or actinic radiation to react the multifunctional (meth)acrylate monomer, prepolymer or oligomer, and polysaccharide by free radical addition polymerization, thereby forming a polymer shell surrounding the droplets of the emulsion.

20. The process according to claim 19 wherein the polysaccharide comprises a moiety with an active hydroxyl, carbon, or acryloyl, and the multifunctional (meth)acrylate comprises a moiety with an unsaturated site for cross-linking of the polysaccharide with the multifunctional methacrylate to form the polymeric shell.

21. The process according to claim 19 wherein the modified polysaccharide in the water phase can function as the emulsifier in the emulsifying step, the emulsifying step being substantially free of other added emulsifier.

22. The process according to claim 19 wherein the multifunctional (meth)acrylate can be selected from a monomer, oligomer or prepolymer having a greater than one ester group on average in the monomer, oligomer or prepolymer, and can be selected from ethylene glycol dimethacrylate, polyethylene glycol dimethacrylate, ethylidene dimethacrylate, trimethylolpropane triacrylate, trimethylolpropane trimethacrylate, pentaerythritol tetraacrylate, tricyclodecane dimethanol dimethacrylate, 1,10 decanediol dimethacrylate, 1,6 hexanediol dimethacrylate, 1,9 nonanediol dimethacrylate, neopentyl glycol dimethacrylate, di-trimethylolpropane tetraacrylate, dipentaerythritol pentaacrylate, ethoxylated (2) bisphenol A dimethacrylate, 2,2 bis[4-(methacyloyl ethoxy) phenyl] propane, ethoxylated (3) bisphenol A diacrylate, dipropylene glycol diacrylate, ethoxylated (4) bisphenol A diacrylate, ethoxylated (4) bisphenol A dimethacrylate, 2,2 bis[4(methacyloyl ethoxy) phenyl] propane, polyethylene glycol trimethylolpropane tri(meth)acrylate, polyester acrylate urethane acrylate and pentaerythritol triacrylate.

23. A process of forming a population of microcapsules, the microcapsules comprising a core material and a shell encapsulating the core material, the shell of the microcapsules formed by an interfacial reaction between a multifunctional (meth)acrylate dispersed in an oil phase, and a polysaccharide dispersed in a water phase, the process comprising:
a. dispersing one or more in oil phases a first initiator, a core material, and a multifunctional (meth)acrylate monomer, prepolymer or oligomer;
b. dispersing in a water phase the polysaccharide, and an optional second initiator;
c. emulsifying the one or more oil phases into the water phase under high shear agitation to form an oil-in-water emulsion comprising droplets of the core material and oil phase monomer dispersed in the water phase;
d. and activating the initiator or initiators by heat or actinic radiation to react the multifunctional (meth)acrylate monomer, prepolymer or oligomer, and polysaccharide by free radical addition polymerization, thereby forming a polymer shell surrounding the droplets of the emulsion, wherein the polysaccharide is of Formula I 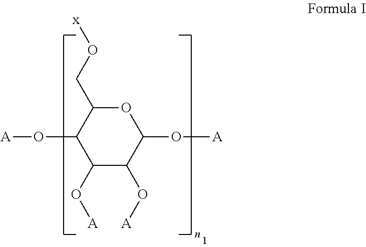
wherein n₁ is an integer from 1 to 1,000, wherein x is a terminal hydrogen or repeating glucose unit of Formula I from 1 to 50 units, and wherein each A are independently a terminal hydrogen, or an adduct selected from the group consisting of Formula II, Formula III, Formula IV, Formula V, and Formula VI, 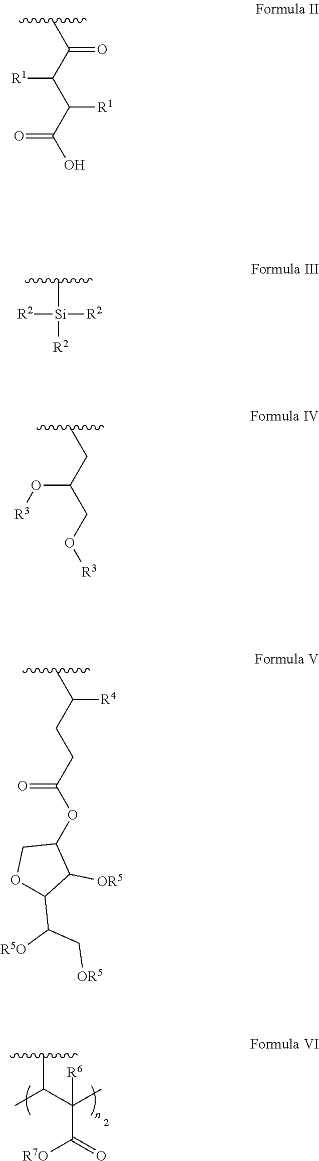
wherein each R¹ are independently a terminal hydrogen, alkyl or alkenyl, from 1 to 20 carbons, each R₂ are independently an alkyl, alkenyl, alkoxy, from 1 to 20 carbons or hydroxy, each R³ are independently a terminal hydrogen, alkyl or alkenyl, from 1 to 20 carbons, R⁴ is an alkyl or alkenyl, from 1 to 20 carbons, R⁵ is a terminal hydrogen, alkyl, alkenyl, from 1 to 20 carbons, or polyoxyethylene, from 1 to 40 carbons, R⁶ is a terminal hydrogen, a hydroxy, an alkoxy, alkyl, alkenyl, alkanol or alkylamine, from 1 to 20 carbons, and R⁷ is a terminal hydrogen, (meth)acrylate, alkyl, alkenyl, alkynyl, amine, amide, arenyl, carboxylate, disulfide, ester, ether, epoxide, isocyanate, or sulfide, from 1 to 20 carbons, and n₂ is an integer from 1 to 1,000.

24. An article of manufacture incorporating the microcapsules according to claim 1.

25. The article of manufacture according to claim 24 wherein the article is selected from the group consisting of a soap, a surface cleaner, a laundry detergent, a fabric softener, a shampoo, a textile, a paper towel, an adhesive, a wipe, a diaper, a feminine hygiene product, a facial tissue, a pharmaceutical, a napkin, a deodorant, a heat sink, a foam, a pillow, a mattress, bedding, a cushion, a cosmetic, a medical device, packaging, an agricultural product, a cooling fluid, a wallboard, and an insulation.
